(19) United States
(12) Patent Application Publication
(10) Pub. No.: US 2015/0088009 A1
(43) Pub. Date: Mar. 26, 2015

(54) SEMI-AUTOMATIC SPHYGMOMANOMETER SYSTEM

(71) Applicant: Koven Technology, Creve Coeur, MO (US)

(72) Inventor: Frank Herndon, Creve Coeur, MO (US)

(21) Appl. No.: 14/483,913

(22) Filed: Sep. 11, 2014

Related U.S. Application Data
(60) Provisional application No. 61/881,362, filed on Sep. 23, 2013.

(51) Int. Cl. A61B 5/0225; A61B 5/0235; A61B 5/022

(52) U.S. Cl. CPC A61B 5/0225; A61B 5/02233; A61B 5/0235
USPC 600/498; 600/499

(57) ABSTRACT

An embodiment of a semi-automatic sphygmomanometer apparatus that provides a means for initiating and terminating inflation of a vascular cuff is disclosed. The semi-automatic sphygmomanometer apparatus includes a motor that operates a pump which generates air pressure to inflate the vascular cuff, wherein operation of the pump is initiated when the inflationary button is depressed and wherein operation of the pump is terminated when the inflationary button is released.

### CROSS REFERENCE TO RELATED APPLICATIONS

This application claims benefit to U.S. provisional patent application Ser. No. 61/881,362 filed on Sep. 23, 2013 and is herein incorporated by reference in its entirely.

### FIELD

This document relates to a sphygmomanometer, and in particular, to a sphygmomanometer with a means for semi-automatic inflation and manual deflation.

### BACKGROUND

A sphygmomanometer is a device used with a vascular cuff and stethoscope for measuring blood pressure in an artery. Typically, a manual sphygmomanometer is connected to a vascular cuff that is wrapped around an arm (or other location as required) of an individual and then inflated to constrict the flow of blood. The vascular cuff is connected to hollow tubing that communicates with a flexible bulb that is manually pumped by the practitioner to cause inflation of the vascular cuff. In addition, a valve and indicator gauge arrangement is interposed between the hollow tubing and the flexible bulb to provide a visual indication of pressure being applied by the vascular cuff and to control the flow of air pressure to the vascular cuff. A stethoscope or Doppler probe is applied over the individual's brachial pulse, which is located on the inside of the individual's upper arm near the elbow (or other location as required), and the valve is then tightened prior to repeatedly pumping the flexible bulb until the vascular cuff reaches a certain pressure, for example 200 mm Hg being shown on the indicator gauge. The vascular cuff is then slowly deflated by loosening the valve or another embodiment, such as an air release trigger. While watching the air pressure fall, the practitioner uses the stethoscope or Doppler probe to listen for when the first audible sound of a heartbeat or pulse of the individual is first heard which provides systolic blood pressure. Once the systolic blood pressure is determined, the valve may be completely opened and the remaining air let out of the vascular cuff. Although such manual sphygmomanometers work well for their intended purpose, the practitioner is still required to manually pump the flexible bulb to inflate the vascular cuff. In addition, certain vascular diagnostic procedures, such as segmental pressures, may require repetitive inflation of several cuffs, often of significant volume, such as an upper thigh cuff, which can lead to repetitive strain injuries.

### BRIEF DESCRIPTION OF THE DRAWINGS

FIG. 1 is a perspective view of a semi-automatic sphygmomanometer system showing the sphygmomanometer apparatus, a vascular cuff, and hollow tubing;

FIG. 2 is a simplified block diagram of the semi-automatic sphygmomanometer system of FIG. 1;

FIG. 3 is a perspective view of the sphygmomanometer apparatus of FIG. 1;

FIG. 4 is a front view of the sphygmomanometer apparatus of FIG. 1;

FIG. 5 is a perspective view of the sphygmomanometer apparatus and related ancillary equipment; and

FIG. 6 is a cross-sectional view of the sphygmomanometer apparatus taken along line 6-6 of FIG. 5.

Corresponding reference characters indicate corresponding elements among the view of the drawings. The headings used in the figures do not limit the scope of the claims.

### DETAILED DESCRIPTION

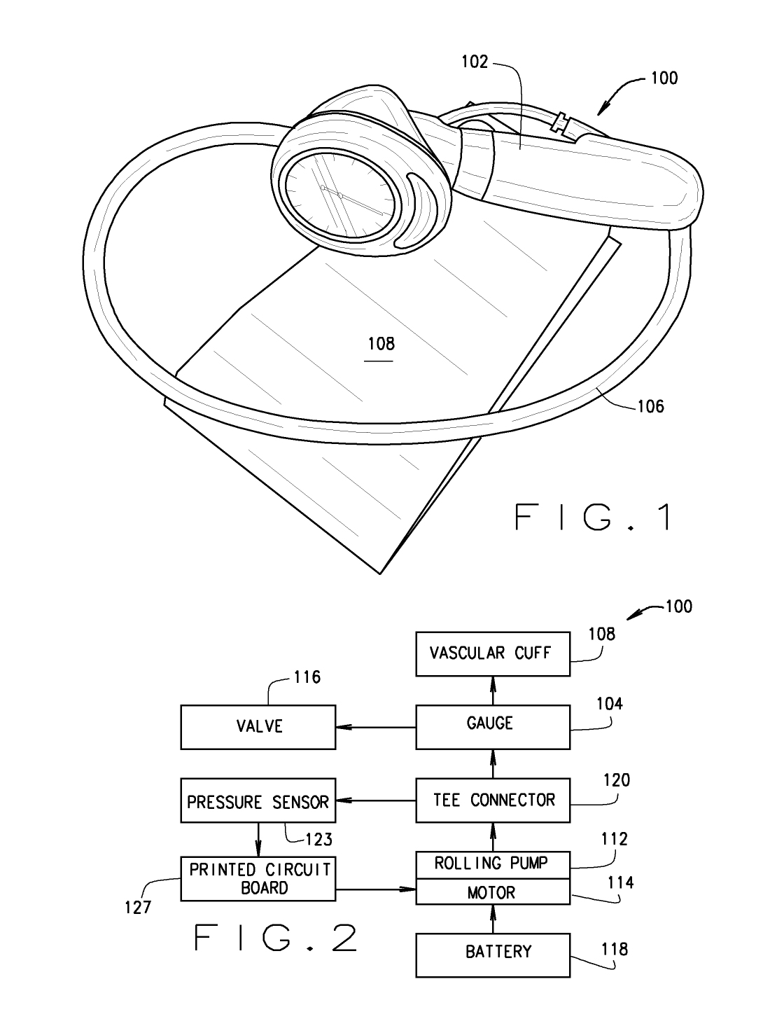

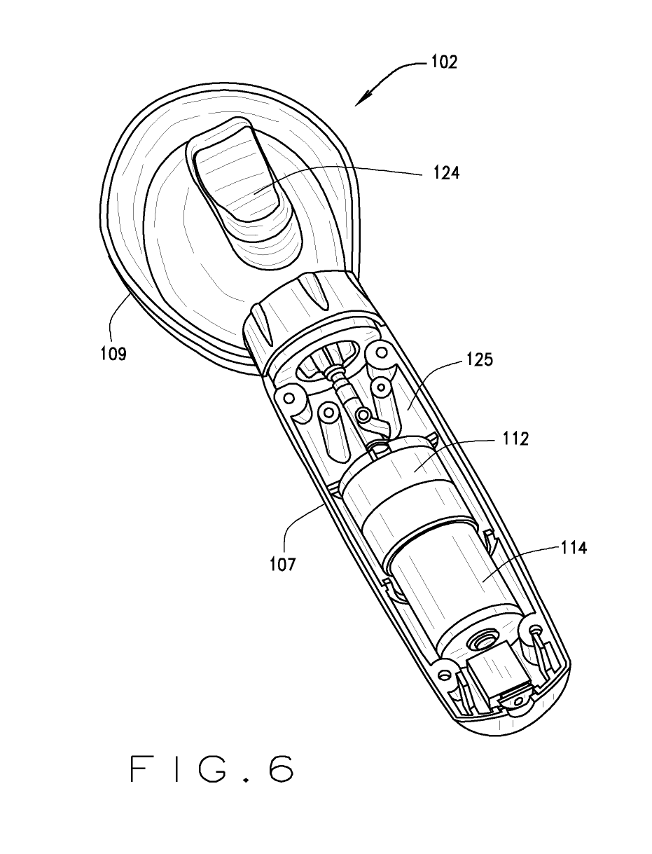

As described herein, a sphygmomanometer system having a means for semi-automatic inflation of a vascular cuff as well as a means for manually terminating inflation of the vascular cuff by a practitioner as an individual's blood pressure is being monitored. In addition, the sphygmomanometer system provides a means for automatically detecting the size of a vascular cuff attached to an individual. Referring to the drawings, an embodiment of a semi-automatic sphygmomanometer system is illustrated and generally indicated as 100 in FIGS. 1-6. As shown in FIGS. 1 and 2, in some embodiments the semi-automatic sphygmomanometer system 100 may include a sphygmomanometer apparatus 102 that is connected to a vascular cuff 108 through hollow tubing 106 in which sphygmomanometer apparatus 102 allows for semi-automatic inflation of the vascular cuff 108 and manual termination of inflation of the vascular cuff 108.

As further shown in FIG. 2, the semi-automatic sphygmomanometer system 100 may include a motor 114 in operative connection to a rolling pump 112 for generating air flow through a tee connector 120. The tee connector 120 receives pressurized air from the rolling pump 112 and splits the pressurize air into two separate air pathways—a first pathway that supplies the pressurized air to an indicator gauge 104, a valve 116 and vascular cuff 108 as well as a second pathway that supplies the pressurized air to a pressure sensor 123.

The pressure sensor 123 is in operative communication with a printed circuit board 127 or other type of processor that receives air pressure data from the pressure sensor 123. In one embodiment, the printed circuit board 127 may automatically detect the size of the vascular cuff 108 connected to the sphygmomanometer apparatus 102. In particular, the sphygmomanometer apparatus 102 provides a means for determining whether the vascular cuff 108 is either a small cuff or a large cuff based on the data received from the pressure sensor 123. The printed circuit board 127 may then adjust the power being supplied to the rolling pump 112 by controlling the amount of power being supplied to the rolling pump 112. For example, if a large vascular cuff 108 is detected by the printed circuit board 127, the rolling pump 112 operates at 100% power, while if a small vascular cuff 108 is detected by the printed circuit board 127 the rolling pump 112 will operate at about 35% power, although other power levels less than 100% are contemplated when a small vascular cuff 108 is used. This arrangement allows the sphygmomanometer apparatus 102 to inflate a vascular cuff 108 at a reasonable inflation speed based on the size of the vascular cuff 108 with a single actuation of the sphygmomanometer apparatus 102.

In some embodiments, the sphygmomanometer apparatus 102 may include a safety feature that automatically terminates inflation of the vascular cuff 108 if the pressure detected by the pressure sensor 123 exceeds a predetermined value, for example 270 mmHg, to prevent over-inflation of the vascular cuff 108.

As shown in FIGS. 1 and 5, the sphygmomanometer apparatus 102 includes an air outlet 111 configured to engage the hollow tubing 106 (FIG. 1), which is connected to the vascular cuff 108 to allow inflation of the vascular cuff 108 by the rolling pump 112. During operation of the pump 112, air flow generated by the pump 112 flows through the hollow tubing 106 to inflate the vascular cuff 108, which is configured to be wrapped around an individual's arm (or other location along the body of an individual) when monitoring an individual's blood pressure as shall be discussed in greater detail below.

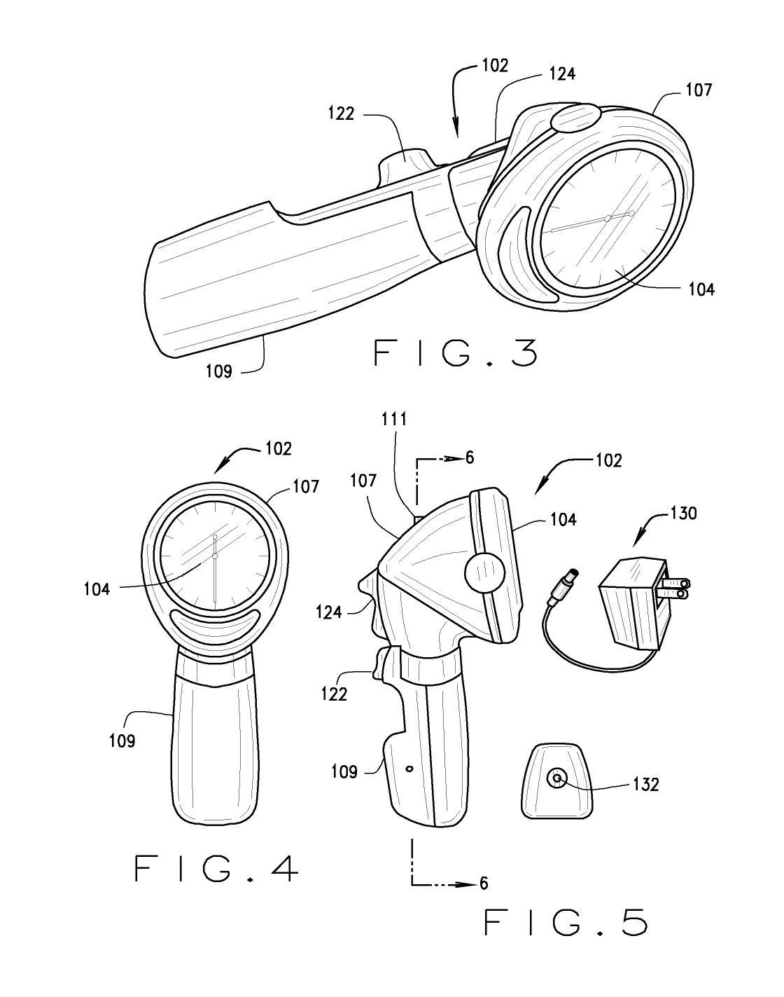

Referring to FIGS. 3-6, the sphygmomanometer apparatus 102 may further include an inflationary button 122 that activates and terminates operation of the motor 114 in order to turn on and off the operation of the rolling pump 112. In operation, the practitioner must depress and maintain the inflationary button 122 in the depressed state to maintain the inflationary button 122 in an “ON” position, which activates the rolling pump 112. Once the practitioner releases the inflationary button 122, the inflationary button 122 is placed in the “OFF” position and the operation of the rolling pump 112 ceases until the practitioner once again depresses the inflationary button 122.

As further shown, the motor 114 may include one or more batteries 118 to provide power to the motor 114 for operating the rolling pump 112. The sphygmomanometer apparatus 102 may include an internal chamber 125 configured to encase the rolling pump 112, motor 114, batteries 118, pressure sensor 123, tee connector 120 and printed circuit board 127. In some embodiments, the sphygmomanometer apparatus 102 be connected directly to an electrical outlet (not shown) through a power adapter 130 (FIG. 5). As shown in FIG. 5, the sphygmomanometer apparatus 102 may include a DC jack 132 that receives an AC adapter 130, which is used to charge one or more batteries 118.

As further shown, the sphygmomanometer apparatus 102 may include a gauge 104 that includes a dial or digital readout for providing a visual indication of air pressure within the vascular cuff 108. The gauge 104 may be connected to a valve 116 that provides selective fluid flow communication between the pump 112, the gauge 104, and the vascular cuff 108 when actuated.

As shown in FIGS. 3 and 5, the sphygmomanometer apparatus 102 may include an air pressure release trigger 124 that allows for the deflation of the vascular cuff 108 when actuated by releasing air pressure from vascular cuff 108. Once inflation of the vascular cuff 108 is terminated by releasing the inflationary button 122, the practitioner may then release the air pressure from the vascular cuff 108 by actuating the air pressure release trigger 124 such that air pressure is released to the atmosphere. In some embodiments, the air pressure release trigger 124 may be a trigger mechanism, a button mechanism, a switch mechanism, or a dial mechanism for releasing air pressure from the sphygmomanometer apparatus 102.

In one method of use of the semi-automatic sphygmomanometer 100, the vascular cuff 108 is wrapped snugly around an individual's arm or other location along the body of the individual. For example, the vascular cuff 108 may be positioned so that the lower part of the vascular cuff 108 is approximately 1 inch above the inner bend of the individual's elbow and over the brachial artery. After inflating the vascular cuff 108 as discussed above until the gauge 104 reads about 200 mm Hg, the practitioner may then place one end of a stethoscope (not shown) over the individual's brachial pulse and actuate the air pressure release trigger 124 to begin deflating the vascular cuff 108. As air pressure falls and the vascular cuff 108 deflates, the practitioner listens carefully for the first audible sound of the individual's heartbeat or pulse through the stethoscope. The first audible sound of the heartbeat or pulse heard through the stethoscope indicates the individual's systolic blood pressure. The remaining air pressure is released from the vascular cuff 108 by actuation of the air pressure release trigger 124. In some embodiments, a Doppler device may be used, rather than a stethoscope, to listen for the heartbeat or pulse.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A semi-automatic sphygmomanometer apparatus comprising:
a motor in operative connection to a pump for generating air pressure;
an inflationary button in operative connection with the motor for activating and terminating operation of the motor, wherein the inflationary button activates operation of the motor when depressed and terminates operation of the motor when not depressed;
an air pressure release trigger for releasing air pressure generated by the pump;
an air outlet in fluid flow communication with the pump;
a hollow tubing having a first end in fluid flow communication with the air outlet; and
an inflatable cuff in fluid flow communication with the hollow tubing for providing air pressure from the pump to the inflatable cuff.

2. The semi-automatic sphygmomanometer apparatus of claim 1, wherein the pump is a rolling pump.

3. The semi-automatic sphygmomanometer apparatus of claim 1, further comprising:
a valve in selective fluid flow communication between the air pressure release trigger and the vascular cuff.

4. The semi-automatic sphygmomanometer of apparatus claim 3, further comprising:
an indicator gauge in fluid flow communication with the valve for providing an indication of air pressure in the vascular cuff.

5. The semi-automatic sphygmomanometer apparatus of claim 4, further comprising:
a tee connector in fluid flow communication with the pump for defining a first pathway that supplies air pressure to the indicator gauge and a second pathway that supplies air pressure to a pressure sensor for detecting the degree of air pressure applied to the vascular cuff.

6. The semi-automatic sphygmomanometer apparatus of claim 1, further comprising:
a pressure sensor for detecting the degree of air pressure applied to the vascular cuff; and
a processor for in operative communication with the pressure sensor for receiving air pressure data indicative of air pressure generated by the pump, wherein the processor determines whether the inflatable cuff is configured to have a large volume or a small volume.

7. The semi-automatic sphygmomanometer apparatus of claim 6, wherein the processor instructs the pump to generate an air pressure based on whether the inflatable cuff is configured to have a large volume or a small volume.

8. The semi-automatic sphygmomanometer apparatus of claim 6, wherein the processor automatically terminates operation of the pump when the air pressure exceeds a predetermined value.

9. The semi-automatic sphygmomanometer apparatus of claim 1, wherein operation of the pump is initiated when the inflationary button is depressed and wherein operation of the pump is terminated when the inflationary button is released.

10. A semi-automatic sphygmomanometer apparatus comprising:
a motor in operative connection to a pump for generating air pressure;
an inflationary button in operative connection with the motor for activating and terminating operation of the motor;
an air pressure release trigger for releasing air pressure generated by the pump;
an air outlet in fluid flow communication with the pump;
a hollow tubing having a first end in fluid flow communication with the air outlet;
an inflatable cuff in fluid flow communication with the hollow tubing for providing air pressure from the pump to the inflatable cuff;
a pressure sensor in operative communication with the pump for detecting the degree of air pressure generated by the pump; and
a processor in operative communication with the pressure sensor for receiving air pressure data indicative of the air pressure generated by the pump, wherein the processor determines whether the inflatable cuff is configured to have a large volume or a small volume.

11. The semi-automatic sphygmomanometer apparatus of claim 10, wherein the processor determines whether the inflatable cuff is configured to have a large volume or a small volume based on data received from the pressure sensor.

12. The semi-automatic sphygmomanometer apparatus of claim 10, wherein the processor instructs the pump to generate an air pressure based on whether the inflatable cuff is configured to have a large volume or a small volume.

13. The semi-automatic sphygmomanometer apparatus of claim 10, wherein the processor automatically terminates operation of the pump when the air pressure exceeds a predetermined value.

14. The semi-automatic sphygmomanometer apparatus of claim 10, further comprising:
a valve in selective fluid flow communication between the air pressure release trigger and the vascular cuff.

15. The semi-automatic sphygmomanometer apparatus of claim 10, wherein operation of the pump is initiated when the inflationary button is depressed and wherein operation of the pump is terminated when the inflationary button is released.

16. The semi-automatic sphygmomanometer of claim 10, further comprising:
an indicator gauge in fluid flow communication with the valve for providing an indication of air pressure in the vascular cuff.

17. The semi-automatic sphygmomanometer apparatus of claim 10, further comprising:
a tee connector in fluid flow communication with the pump for defining a first pathway that supplies air pressure to the gauge and a second pathway that supplies air pressure to a pressure sensor for detecting the degree of air pressure applied to the vascular cuff.
